(19) United States
(12) Patent Application Publication
(10) Pub. No.: US 2016/0221890 A1
(43) Pub. Date: Aug. 4, 2016

(54) SILP CATALYST FOR HYDROFORMYLATION OF OLEFINS WITH SYNTHESIS GAS

(71) Applicant: EVONIK DEGUSSA GMBH, Essen (DE)

(72) Inventors: Dirk FRIDAG, Haltern am See (DE); Hanna HAHN, Duisburg-Baerl (DE); Katrin Marie DYBALLA, Recklinghausen (DE); Robert FRANKE, Marl (DE); Simon WALTER, Erlangen (DE); Marco HAUMANN, Velden (DE); Peter WASSERSCHEID, Erlangen (DE)

(73) Assignee: EVONIK DEGUSSA GMBH, Essen (DE)

(21) Appl. No.: 15/003,449

(22) Filed: Jan. 21, 2016

(30) Foreign Application Priority Data
Jan. 29, 2015 (DE) 102015201560.2

(51) Int. Cl. C07B 41/06; C07F 15/00; C07C 45/50; B01J 31/02; B01J 31/18

(52) U.S. Cl. CPC C07B 41/06; B01J 31/0292; B01J 31/185; C07C 45/50; C07F 15/0073; B01J 2231/321; B01J 2531/822

(57) ABSTRACT

SILP catalyst for the hydroformylation of olefins with synthesis gas, wherein the catalyst comprises at least one spherical catalyst pellet of radius R, characterized in that the catalyst complies with the following SILP modulus:

$$\frac{{c}_{i}}{{c}_{i , S}} = \frac{R}{x} \frac{sinh ( {φ}_{SILP} \frac{x}{R} )}{sinh ( {θ}_{SILP} )}$$

where φ_{SILP} is a dimensionless number in respect of the spherical catalyst pellet relating the mass transfer, in the pore, across the phase interface between gas and ionic liquid and in the ionic liquid to the reaction, where cᵢ is the concentration of a component i and c_{i,S} is the concentration of a component i at the surface of the SILP catalyst pellet, and x is the position.

Also featured is a process for hydroformylation of olefins with synthesis gas by using the SILP catalyst.

The present invention relates to an SILP catalyst for hydroformylation of olefins with synthesis gas and also to a process for hydroformylating olefins with synthesis gas by using said catalyst.

The reaction of olefin compounds with a mixture of carbon monoxide and hydrogen in the presence of a catalyst to form aldehydes is known as hydroformylation/oxonation or as Roelen reaction:

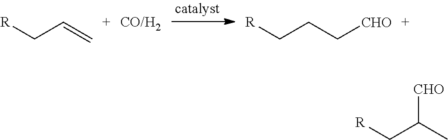

FIG. 1 Illustration A: Reaction Scheme for Hydroformylation of Olefins

Homogeneous type catalyst complexes for the hydroformylation reaction generally have the generic structure HₓM_{y}(CO)_{z}Lₙ, where M is a transition metal atom capable of forming metal carbonyl hydrides and L is one or more than one ligand.

Preferred transition metals for use in the aforementioned catalyst complexes are cobalt and rhodium, while ruthenium, iridium, palladium, platinum and iron are less preferable. Starting with the discovery of phosphane-modified catalysts in 1968, ligand-modified cobalt and rhodium catalysts have come to represent the state of the art in hydroformylation.

There are various industrial hydroformylation processes, of which the Ruhrchemie/Rhone-Poulenc process represents a milestone in the development of industrial hydroformylation. In this process, the catalyst system consists of rhodium and a water-soluble ligand and is dissolved in an aqueous phase, while the reactant-product mixture forms a second liquid phase. After the two phases have been mixed together by stirring and the olefin, if gaseous, as well as synthesis gas has been passed through the mixture, the reactant-product mixture is phase-separated from the catalyst system and worked up by distillation.

One disadvantage with this process is that rhodium losses due to leaching out are unavoidable, which is problematic given rhodium prices. A further disadvantage is that the spectrum of usable short-chain olefins is limited. This is because the solubility of olefins in the aqueous catalyst phase decreases with increasing chain length, and so reduces the reaction rate. This effect can only marginally be compensated by intensive stirring, so it is impossible to ensure economically viable practice of the process for >C4.

The idea behind the development of alternative processes, driven in particular by the very high rhodium prices, is to immobilize the rhodium-based catalyst systems hitherto a homogenous presence in the reaction mixture. In fact, what is generally referred to as immobilization is the “simple” act of segregating the catalyst species from the reactant-product phase without any additional process of separation. Immobilization concepts are designed to maintain the catalytically active species in a different phase from the reactants/products.

### BRIEF DESCRIPTION OF DRAWINGS

FIG. 1 contains an overview of immobilization concepts for homogeneous catalysts.

FIG. 2 shows a further SILP concept employed in heterogenized catalyst complexes.

FIG. 3 illustrates a model for porous spherical SILP catalyst pellet.

FIG. 4 shows, a concentration gradient in IL film as a function of mass transfer and reaction after Lewis and Whitman.

FIG. 5 depicts the pore utilization rate as a function of the resulting modified Thiele moduli against the loading with ionic liquid.

FIGS. 6a and 6b show the influence of the solubility, expressed by the Henry coefficient, and the porosity of the support for case scenario III.

FIG. 7 depicts the efficiency against the loading with ionic liquid as a function of mass transfer area for the SILP modulus φ_{SILP,I}.

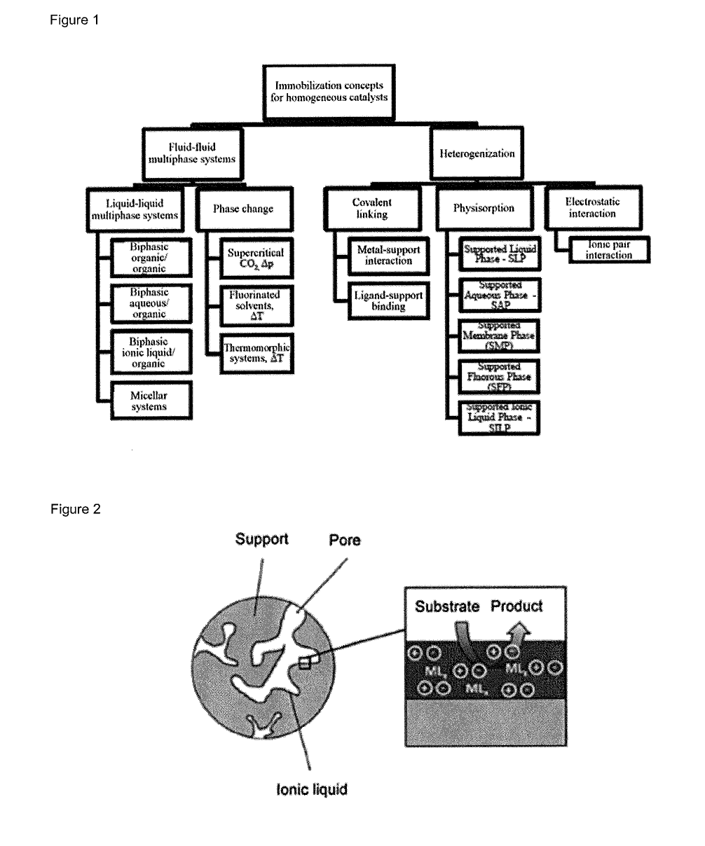

FIG. 1 contains an overview of immobilization concepts for homogeneous catalysts.

There are numerous approaches to immobilizing homogeneous catalysts found in the literature (see for instance E. Lindner, T. Schneller, F. Auer, H. A. Mayer, —Chemistry in Interphases—A New Approach to Organometallic Syntheses and Catalysis, Angew. Chem., Int. Ed. 1999, 38, 2154-2174 and D. J. Cole-Hamilton, R. P. Tooze, Catalyst Separation, Recovery and Recyling, Springer, 2006). Homogeneous catalyst complexes can be enclosed in a polymer matrix; attached to the support via ionic or covalent bonds; linked to the support via hydrogen bonds; or be present on the support as a solution in a thin physisorbed film of a fluid. These possibilities all have their advantages and disadvantages. Catalyst leaching represents the greatest problem absent an ionic or covalent link between catalyst and support. For a covalent link, the ligand has to be specially prepared and leaching is in any case only avoided when the link is sufficiently strong. For an ionic link, the catalyst complex likewise has to bear a charge, but the stability may be affected by counter-ions formed in reactions involving possible salt formation. For a link via hydrogen bonding, the ligand is in competition with the reactants, products and solvents.

In addition to the supported aqueous phase (SAP) concept, which is unsuitable for hydrolysis-sensitive ligands, however, the supported liquid phase (SLP) concept represents a further concept for heterogenization of homogeneous catalyst complexes. Supported liquid phase (SLP) catalysts are based on the physisorption of a solvent on the surface of a porous support while a homogeneous catalyst complex is a solute in the solvent. Among the liquid phases used for the hydroformylation reaction are molten salts such as, for example, triphenylphosphane (TPP). Here TPP serves as a ligand as well as a solvent for the catalyst complex, and therefore is used in large excess. Yet a large excess of ligand disadvantageously entails the formation of various transition metal complexes, which may serve to reduce the catalytic activity.

The supported ionic liquid phase (SILP) concept, as seen in FIG. 2, is a further concept employed in heterogenized catalyst complexes (source: R. Fehrmann, A. Riisager, M. Haumann, Supported Ionic Liquids: Fundamentals and Applications, Wiley-VCH, 2014). A supported ionic liquid phase (SILP) catalyst consists of a catalytically active metal complex, an ionic liquid and a supporting material. The catalyst complex is a solute in the ionic liquid which ideally forms an immobilized thin film on the pore walls of the support. What is obtained in macroscopic terms is a heterogeneous catalyst whose activity and selectivity are similar to those of a homogeneous catalysis under comparable reaction conditions.

The problem addressed by the present invention in relation to the known prior art is that of developing a model to describe the mass transfer and reaction in SILP catalysts. This shall be the starting point for providing alternative catalysts for SILP catalysis, in particular for SILP-catalysed hydroformylation of olefins with synthesis gas, and also a process for SILP-catalysed hydroformylation of olefins with synthesis gas by using these catalysts.

This problem is solved according to the invention by an SILP catalyst and also by a process for hydroformylation of olefins with synthesis gas by using an SILP catalyst having the features of the claims, and in particular by an SILP catalyst which comprises at least one spherical catalyst pellet of radius R and which complies with the following SILP modulus:

$$\frac{{c}_{i}}{{c}_{i , S}} = \frac{R}{x} \frac{sinh ( {φ}_{SILP} \frac{x}{R} )}{sinh ( {θ}_{SILP} )} \qquad ( Eq . 1 )$$

where φ_{SILP} is a dimensionless number in respect of the spherical catalyst pellet relating the mass transfer, in the pore, across the phase interface between gas and ionic liquid and in the ionic liquid to the reaction,

where cᵢ is the concentration of a component i and c_{i,S} is the concentration of a component i at the surface of the SILP catalyst pellet, and x is the position.

The hereinbelow described model, which describes the mass transfer of a gaseous component and the reaction in an SILP catalyst, rests on Thiele's modulus as described in the literature and on the two-film model of Lewis and Whitman (see: E. W. Thiele, ‘Relation between Catalytic Activity and Size of Particle’, Ind. Eng. Chem. 1939, 31, 916-920 and W. K. Lewis, W. G. Whitman, ‘Principles of Gas Absorption’, Ind. Eng. Chem. 1924, 16, 1215-1220).

The activity of an SILP catalyst is not just influenced by the nature of the homogeneous precious metal complexes; the distribution of the complexes and of the ionic liquid (IL) likewise plays a part.

The activity is further influenced by the pore radius distribution, the wetting behaviour of the ionic liquid, the proportion of the IL in the pore network, the solubility of the substrates and products in the IL, the interactions of precious metal complex and support and/or of complex and IL and optionally also the formulation of the SILP catalyst. Investigations into the interactions of the ionic liquid [BMIM][NTf2] with an ideal planar surface on an alumina support showed continuous film formation in the nanometre region (see: M. Sobota, I. Nikiforidis, W. Hieringer, N. Paape, M. Happel, H.-P. Steinruck, A. Gorling, P. Wasserscheid, M. Laurin, J. r. Libuda, ‘Toward Ionic-Liquid-Based Model Catalysis: Growth, Orientation, Conformation, and Interaction Mechanism of the [Tf2N]-Anion in [BMIM][Tf2N] Thin Films on a Well-Ordered Alumina Surface’, Langmuir 2010, 26, 7199-7207). Haumann et al. were able to show by means of solid state NMR measurements that islands of ionic liquid form on real pore walls at loadings below 10% by volume (see: M. Haumann, A. Schönweiz, H. Breitzke, G. Buntkowsky, S. Werner, N. Szesni, ‘Solid-State NMR Investigations of Supported Ionic Liquid Phase Water-Gas Shift Catalysts: Ionic Liquid Film Distribution vs. Catalyst Performance’, Chem. Eng. Technol. 2012, 35, 1421-1426. At higher loading, no signal was detected for free silanol groups on the surface of the support. From this it can be inferred that a monomolecular film forms on the surface of the support, but not whether the film grows in a continuous homogeneous manner. In respect of the gas/IL phase interface, it has been shown that the orientation of the alkyl chain on the cation of the IL has an effect on the structure of the phase interface and thus on the solubility and the phase transfer. However, the interplay of all these effects is very complicated and has so far not been resolved in detail. The following assumptions are accordingly made for the model:
The fluid is gaseous or liquid, not a mixture of two phases.
The catalyst pellet is isothermal, temperature effects are not taken into account.
The support is considered to be inert and diffusion of reacting gases and products only takes place inside the pore.
The pores are interconnected.
Film diffusion through the outer boundary layer of the pellet is very rapid as compared with diffusion in the pore.
Mass transfer solely takes place on account of diffusion.
The reaction is first order and the retroreaction is not taken into account.

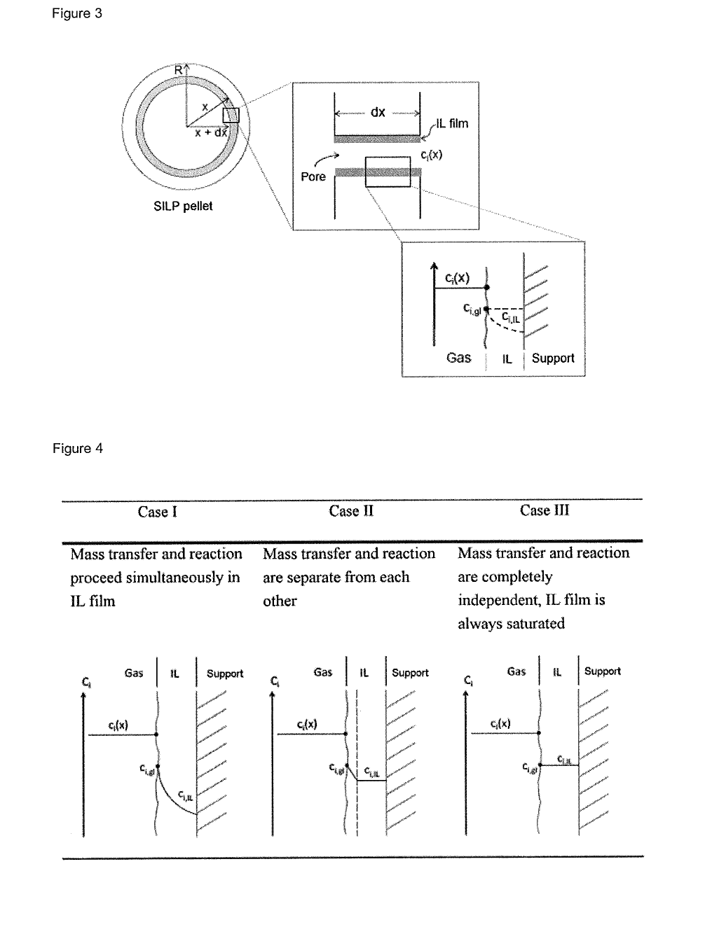

FIG. 3 illustrates the model. The balancing space is formed by a porous spherical SILP catalyst pellet. Described are the mass transfer inside a pore filled with ionic liquid and the mass transfer across the gas/IL phase interface with subsequent reaction.

It can be assumed to a first approximation that there is no concentration gradient in the IL film. This assumption is certainly correct for small loadings where film thickness is in the nanometre range. Ionic liquids have a viscosity which is two or three times higher than that of organic solvents. A higher viscosity results in a smaller diffusion coefficient in the liquid phase. For carbon dioxide, which has a high solubility in ILs which is comparable to organic solvents, the diffusion coefficient in [EMIM][NTf2] is reduced tenfold as compared with toluene. Three case scenarios are accordingly distinguished in the model (cf. FIG. 4: Concentration gradient in IL film as a function of mass transfer and reaction after Lewis and Whitman, source: W. K. Lewis, W. G. Whitman, ‘Principles of Gas Absorption’, Ind. Eng. Chem. 1924, 16, 1215-1220.):

Case scenario I: Reaction and mass transfer take place simultaneously in the IL film.

Case scenario II: The reaction is slow as compared with the mass transfer.

Case scenario III: Mass transfer is significantly faster than the reaction owing to the thin IL film.

The initial formula is formed by the general material balance of a material i expanded to include a term to describe the gas/IL phase transfer:

$$\frac{\partial {c}_{t}}{\partial t} = - div ( {c}_{i} \vec{u} ) + div ( {D}_{i} grad {c}_{i} ) + {R}_{i} \pm a {β}_{i} ( {c}_{i} - {c}_{i , gl} ) \qquad ( Eq . 2 )$$

Convective flow has been ruled out from the start in the assumptions. It follows that the velocity u=0. The dispersion coefficient Dᵢ in the gas phase is position independent and is hereinbelow referred to as effective diffusion coefficient D_{eff}. So for one dimension the result is:

$$\frac{\partial {c}_{i}}{\partial t} = {D}_{eff} d \frac{d c}{d x} + {R}_{i} + a {β}_{i} ( {c}_{i} - {c}_{i , gl} ) \qquad ( Eq . 3 )$$

For a spherical pellet of radius R, the material balance for a constant-volume irreversible reaction under the incipitly formulated assumptions follows from the general material balance for monophasic reaction systems. Applying ∂cᵢ/∂t=0 (steady state conditions) and Rᵢ=vᵢⱼYⱼ=−1kc_{i,1L}, the material balance reads as follows:

$${D}_{eff} ( \frac{{d}^{2} {c}_{i}}{d {x}^{2}} + \frac{2}{x} \frac{d {c}_{i}}{d x} ) - ( - {kc}_{i , IL} \pm a {β}_{i} ( {c}_{i , IL} - {c}_{i , gl} ) ) = 0 \qquad ( Eq . 4 )$$

The effective diffusion coefficient of the gaseous component in the empty pore is described via the following relation:

$${D}_{eff}^{0} = \frac{{D}_{i , g} {ɛ}^{0}}{{τ}^{0}} \text{/} {m}^{2} {s}^{- 1} \qquad ( Eq . 5 )$$

Equation 5 shows that the effective diffusion coefficient depends on the porosity and tortuosity of the support. Both factors in an SILP catalyst are additionally influenced by the loading a with ionic liquid. The loading α is a volume-based quantity. It is therefore possible to state the following relation for the porosity:

ε=εₚ(1−α) (Eq. 6)

The tortuosity is a concept describing the ratio of mean pore length (Lₑ) to the length (L) wherealong the diffusion stream is defined.

τ=Lₑ/L (Eq. 7)

Numerous correlations have been reported in the literature for establishing a relation between porosity and tortuosity. The following relation has been found to be particularly accurate:

τ=1pln ε (Eq. 8)

The parameter p here is a tuning parameter which is modified by physical effects, for example the hygroscopicity of the support. This relation shall likewise be included in this model to describe the effective diffusion coefficient. The parameter p describes the homogeneity of IL film formation in this model:

τ=1−plnε°(1−α), (Eq. 9)

where p=1 (ideal film formation), p→0 (island formation and/or flooding of pores and film formation).

Having regard to Equation 6 and Equation 9, the effective diffusion coefficient depends on the loading α as follows:

$${D}_{eff} = \frac{{D}_{i , g} {ɛ}^{0} ( 1 - α )}{1 - p ln {ɛ}_{P} ( 1 - α )} \text{/} {m}^{2} {s}^{- 1} \qquad ( Eq . 10 )$$

By introducing the relations:

H₁₂=cᵢ/c_{i,g1}>l.with H₁₂=Henry coefficient/−. (Eq. 11)

Introducing dimensionless quantities:

y=x/R. (Eq. 12)

f=cᵢ/c_{i,S} (Eq. 13)

Equation 4 yields

$$\frac{2}{y} \frac{d f}{d y} + \frac{{d}^{2} f}{d {y}^{2 }} - {φ}_{SILP} f = 0 \qquad ( Eq . 14 )$$

where φ_{SILP} is a dimensionless number in respect of a spherical SILP particle relating the mass transfer, in the pore, across the gas/IL phase interface and in the IL to the reaction. The following moduli φ_{SILP} were derived as a function of the contemplated concentration profiles in the IL film:

TABLE 1
Modified Thiele moduli for a spherical SILP catalyst pellet as a function of mass transfer in
the IL film

| Case I | Case II | Case III |
|---|---|---|
| ${φ}_{SILP , I} = R \sqrt{\frac{\sqrt{{kD}_{i}^{IL}} a}{{D}_{eff} {H}_{12}}}$ | ${φ}_{SILP , II} = R \sqrt{\frac{2 k {β}_{i} a}{{D}_{eff} {H}_{12} ( k + {β}_{i} a )}}$ | ${φ}_{SILP , III} = R \sqrt{\frac{k}{{D}_{eff} {H}_{12}}}$ |

The following relation for the mass transfer area is defined for an ideal cylindrical sample from a geometric dependence:

α=α°\sqrt{1−α} (Eq. 15)

Applying the boundary conditions

$$\begin{matrix}( I ) y = 1 f = 1 & ( Eq . 16 ) \\ ( II ) y = 0 \frac{d f}{d y} = 0 ( Symmetriebedingung ) & ( Eq . 17 )\end{matrix}$$

Equation 14 yields the following solution:

$$f = \frac{1}{y} \frac{( {e}^{{φ}_{SILP} Y} - {e}^{- {φ}_{SILP} Y} )}{( {e}^{{φ}_{SILP}} - {e}^{- {φ}_{SILP}} )} = \frac{1}{y} \frac{sinh ( {φ}_{SILP} Y )}{sinh ( {φ}_{SILP} )} \qquad ( Eq . 18 )$$

Resubstitution of Equation 18 yields the ratio of the concentration cᵢ to the concentration at the pellet surface c_{i,S} as a function of the position x:

$$\frac{{c}_{i}}{{c}_{i , S}} = \frac{R}{x} \frac{sinh ( {φ}_{SILP} \frac{x}{R} )}{sinh ( {φ}_{SILP} )} \qquad ( Eq . 19 )$$

Equation 19 can be used to define an effective reaction rate r_{eff} based on the reaction volume V_{R}. The ratio of effective to intrinsic reaction rate yields the catalyst efficiency and/or pore utilization rate of an SILP pellet:

$$\begin{matrix}η = \frac{{( {r}_{eff} )}_{{V}_{R}}}{{r}_{intr }} & ( Eq . 20 ) \\ {r}_{eff , {V}_{R}} = \frac{1}{{v}_{k}} {\int }_{0}^{R} r ( x ) 4 π {x}^{2} d x & ( Eq . 21 ) \\ r ( x ) = {k}_{{V}_{IL}} \cdot {c}_{i} & ( Eq . 22 ) \\ {V}_{K} = \frac{4}{3} π {R}^{3} & ( Eq . 23 ) \\ {η}_{P} = \frac{{r}_{eff}}{r} = \frac{3}{{φ}_{SILP}} ( \frac{1}{tanh ( {φ}_{SILP} )} - \frac{1}{{φ}_{SILP}} ) & ( Eq . 24 )\end{matrix}$$

Similarly to the interpretation of the Thiele modulus, values <3 for the SILP moduli lead to an efficiency near 100%.

The moduli will now be discussed in relation to a model reaction A→B. The influence of any one parameter in the moduli must always be viewed in relation to the other parameters and not in isolation.

FIG. 5 depicts the pore utilization rate as a function of the resulting modified Thiele moduli against the loading with ionic liquid. The highest pore efficiency is obtained for case scenario III, where there is no concentration gradient in the IL film. Case scenario I exhibits the lowest efficiency as a function of the loading.

The trajectories reflect the assumptions made for the respective case scenarios. A pronounced concentration decrease in the IL film results in an increased mass flow within the film. As a result of the assumption, the reaction in the liquid phase is faster than the mass flow into the film. The slowed mass transfer across the gas/IL phase interface relative to the reaction leads to a poorer utilization of the SILP catalyst pellet having regard to the effective diffusion coefficient. To achieve an increase in the efficiency, the effective diffusion coefficient would have to be increased by choosing a different support and/or pore microstructure or the solubility of the reactants in the ionic liquid would have to be increased dramatically.

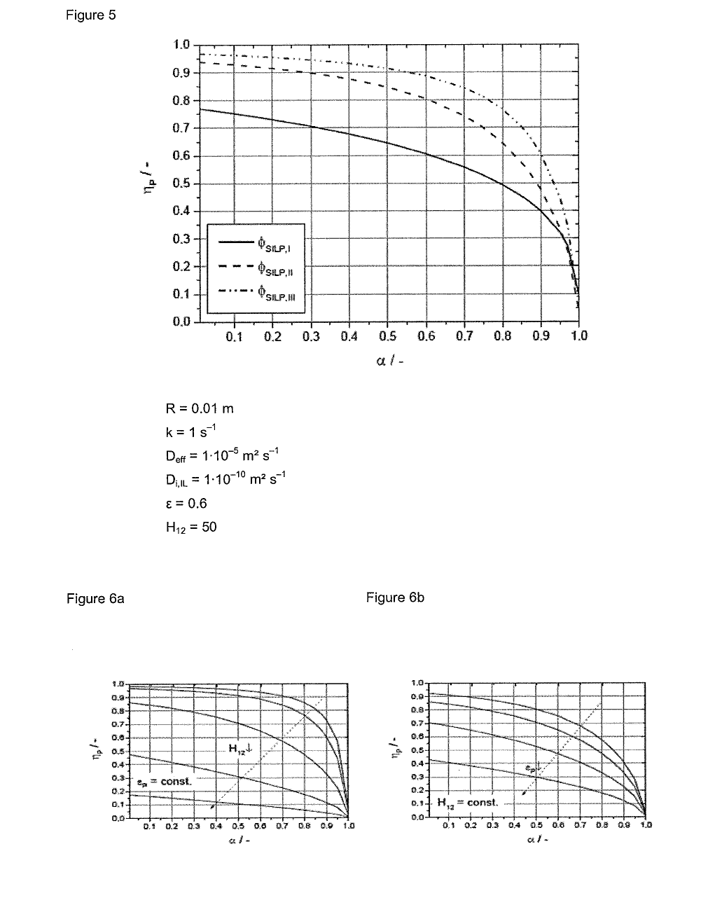

The influence of the solubility, expressed by the Henry coefficient, and the porosity of the support is depicted in FIGS. 6a and 6b for case scenario III.

The catalyst efficiency is always found to decrease with increasing loading. This behaviour is associated with the decreasing effective diffusion coefficient in the narrowing pore. The limiting cases of α=1 and α=0 are not considered in this model. It is maybe theoretically possible in some cases to achieve a higher efficiency with a loading of α=1 than with a loading of α<1. FIG. 6a depicts the efficiency of an SILP catalyst pellet versus the loading with ionic liquid as a function of the Henry coefficient under the conditions of SILP modulus type III φ_{SILP,III}. The Henry coefficient varies in the range H₁₂=1−1000. The Henry coefficient is an IL-specific quantity. As the Henry coefficient decreases, the solubility of the substrate in the ionic liquid increases. As a result, the catalyst efficiency decreases with increasing substrate solubility given a correspondingly small effective diffusion coefficient.

FIG. 6b depicts the efficiency of an SILP catalyst pellet versus the loading with ionic liquid as a function of the porosity of the support under the conditions of SILP modulus type III φ_{SILP,III}. Catalyst efficiency decreases with increasing pore volume. However, as the porosity increases, there is also an increase in the mass transfer area, promoting a fast reaction.

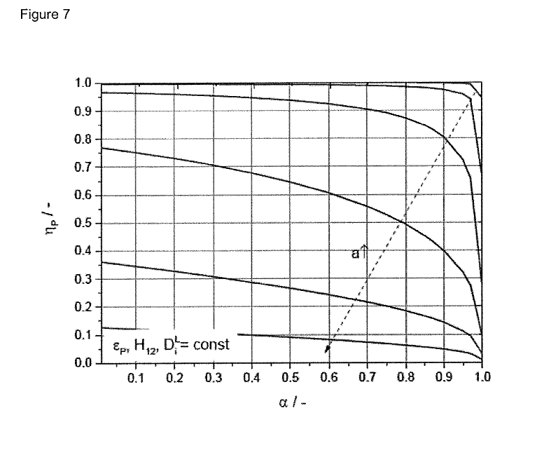

Care should be taken to ensure that the absolute amount of ionic liquid leads to different film thicknesses as a function of porosity. Similar efficiencies are ultimately achievable thereby. The optimum porosity of a support for SILP catalysis is thus influenced by many factors. In the case of a strongly exothermic and rapidly proceeding reaction, for example, a low porosity and a high loading can lead to improved removal of heat from the catalyst bed.

Where the mass transfer across the gas/IL phase interface and a rapid reaction take place simultaneously, the SILP modulus φ_{SILP,I} is obtained. FIG. 7 depicts the efficiency against the loading with ionic liquid as a function of mass transfer area for the SILP modulus φ_{SILP,I}-.

When there is a concentration gradient in the IL film, the mass transfer area is of decisive importance for the utilization of the pellet. The Henry coefficient is set to a constant value of H₁₂=10 in FIG. 7. The specific mass transfer area is varied in the range from 10² m⁻¹ to 10¹⁰ m⁻¹. It is further assumed that the IL film grows homogeneously across the cross section through a cylindrical pore as the loading increases. A large mass transfer area promotes a rapid conversion of the reactants. Where a costly catalyst complex is employed, an excessively large transfer area leads to a poor degree of efficiency and ultimately to an uneconomic utilization of the catalyst. Literature values reported for the diffusion coefficient D_{I,IL} of gases, for example

hydrogen, carbon dioxide, ethene or propene, in ionic liquids show a negligible influence on the efficiency as a function of the loading.

Case scenario II contemplates the mass transfer and the reaction separately from each other, and therefore not only the Henry coefficient and the diffusion coefficient but also the formal “boundary layer thickness” δ feature in the model. The influence of the mass transfer coefficient β on the efficiency is quite minimal and therefore will not be more particularly described.

The three case scenarios all show that it is the solubility of the substrates and the effective diffusion coefficient which have the greatest influence on the efficiency. Improved diffusion in the pore can be accomplished either through the morphology of the support or by raising the diffusion coefficient, for example by raising the temperature.

The models further show that an increased solubility does lead to an increased reaction rate, but not necessarily to better utilization of the pellet.

Catalysts in accordance with the present invention were obtained by admixing porous supporting materials with a solution comprising a catalyst complex, an ionic liquid and a stabilizer.

Dichloromethane was the preferred solvent used here. Deposition of the components in the pore network of the support was preferably effected by concentrating in a rotary evaporator. After solvent removal, the catalyst is in the form of a free-flowing powder.

The hydroformylation was carried out with particular preference in the gas phase, and the synthesis gas used was a mixture of carbon monoxide (Linde AG, 3.7) and hydrogen (Linde AG, 5.0). The olefin used comprised specifically short- to medium-chain unsaturated hydrocarbons, e.g. 1-butene or 2-butene.

Preferred catalyst complexes employed for the synthesis of SILP catalysts are itemized below in Table 2.

TABLE 2
Overview of employed precursors and homogeneous catalysts

| | | Molecular | |
|---|---|---|---|
| | | weight | CAS |
| Complex | Abbreviation | g mol⁻¹ | number |
| (acetylacetonate)dicarbonyl- | Rh(CO)₃(acac) | 258.03 | 14874-82-9 |
| rhodium(I) | | | |
| (acetylacetonate)(1,5- | Rh(acac)(COD) | 310.19 | 12245-39-5 |
| cyclooctadiene)rhodium(I) | | | |
| tris(triphenylphosphine)- | RhCl(PPh₃)₃ | 925.22 | 14694-95-2 |
| rhodium(I) chloride | | | |
| rhodium(III) chloride | RhCl₃ | 209.26 | 10049-07-7 |

Preferred ionic liquids employed for the synthesis of SILP catalysts are itemized below in Table 3.

TABLE 3
Physical constants of ionic liquids used

| | | Molar mass/ | Density¹/ | Viscosity¹/ | Viscosity²/ |
|---|---|---|---|---|---|
| Ionic liquid | Abbreviation | g mol⁻¹ | g cm⁻³ | mPa s | mPa s |
| 1-ethyl-3- | [EMIM] | 391.31 | 1.530 | 30.63 | 6.96 |
| methylimidazolium | [NTf₂] | | | | |
| bis(trifluoromethyl- | | | | | |
| sulphonyl)imide | | | | | |
| 1-butyl-3- | [BMIM] | 419.36 | 1.40 | 32.46 | 7.15 |
| methylimidazolium | [NTf₂] | | | | |
| bis(trifluoromethyl- | | | | | |
| sulphonyl)imide | | | | | |
| 1-methyl-3 - | [OMIM] | 475.47 | 1.37 | 38.5 | 7.34 |
| octylimidazolium | [NTf₂] | | | | |
| bis(trifluoromethyl- | | | | | |
| sulphonyl)imide | | | | | |
| 1-benzyl-3- | [BzMIM] | 453.38 | 1.443 | 124.76 | 7.16 |
| methylimidazolium | [NTf₂] | | | | |
| bis(trifluoromethyl- | | | | | |
| sulphonyl)imide | | | | | |
| 1-benzyl-3- | [BzEIM] | 467.41 | 1.453 | 91.64 | 6.65 |
| ethylimidazolium | [NTf₂] | | | | |
| bis(trifluoromethyl- | | | | | |
| sulphonyl)imide | | | | | |
| 1-benzyl-3- | [BzBIM] | 495.46 | 1.346 | 139.63 | 7.44 |
| butylimidazolium | [NTf₂] | | | | |
| bis(trifluoromethyl- | | | | | |
| sulphonyl)imide | | | | | |
| 1,3-dibenzylimidazolium | [BzBzIM] | 529.48 | | solid | 8.43 |
| bis(trifluoromethyl- | [NTf₂] | | | | |
| sulphonyl)imide | | | | | |

¹at 25° C.
²at 100° C.

Preferred ligands used for the synthesis of SILP catalysts include, for one, the diphosphite ligand 2,2′-(3,3′-di-tert-butyl-5,5′-dimethoxybiphenyl-2,2′-diyl)bis(oxy)bis/4,4,5,5-tetraohenyl-1,3,2-dioxaphospholane), which is depicted in Illustration B and which by virtue of its substructure is also known as benzopinacol (BzP).

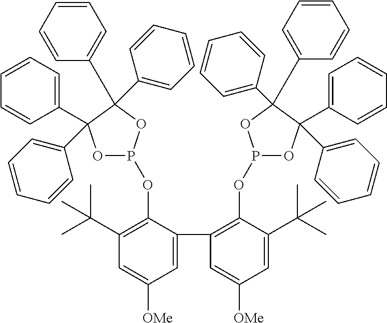 [SMILES: COc1cc(-c2cc(OC)cc(C(C)(C)C)c2OP2OC(c3ccccc3)(c3ccccc3)C(c3ccccc3)(c3ccccc3)O2)c(OP2OC(c3ccccc3)(c3ccccc3)C(c3ccccc3)(c3ccccc3)O2)c(C(C)(C)C)c1]

### Illustration B: Benzopinacol Ligand

Another preferred ligand is the ligand BIPHEPHOS (6,6′-[(3,3′-di-tert-butyl-5,5′-dimethoxy-1,1′-biphenyl-2,2′-diyl)bis(oxy)]bis(dibenzo[d,f][1,3,2]-dioxaphosphepine)), shown in Illustration C.

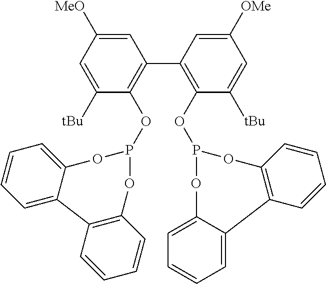 [SMILES: COc1cc(-c2cc(OC)cc(C(C)(C)C)c2Op2oc3ccccc3c3ccccc3o2)c(Op2oc3ccccc3c3ccccc3o2)c(C(C)(C)C)c1]

### Illustration C: BIPHEPHOS (6,6′-[(3,3′-di-tert-butyl-5,5′-dimethoxy-1,1′-biphenyl-2,2′-diyl)bis(oxy)]bis(dibenzo[d,f][1,3,2]-dioxaphosphepine))

One suitable supporting material for the SILP catalysts of the invention is Silica Gel 100, especially with a particle size of 0.063 to 0.2 mm. Suitable supporting materials further include industrial extrudates based on SiO₂, for example granular supports, in particular with a particle size of 1 to 2 mm or rod-shaped supports of any desired length and from, for example, 1 to 2 mm, e.g. 1.6 mm, in diameter.

A preferred stabilizer has the structure—shown in Illustration D—of the sterically hindered diamine bis(2,2,6,6-tetramethyl-4-piperidyl) sebacate.

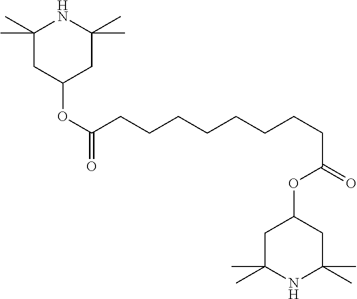

### Illustration D: Bis(2,2,6,6-tetramethyl-4-piperidyl) sebacate

The aforementioned catalyst complexes, ionic liquids, ligands, supporting materials and stabilizers are freely combinable to prepare SILP catalysts that are in accordance with the present invention.

1. Process for hydroformylation of olefins with synthesis gas by using an SILP catalyst comprising at least one spherical catalyst pellet of radius R, characterized in that the ratio of the concentration of a component i to its concentration at the surface of the SILP catalyst pellet as a function of the position x is defined by the following relation:
$$\frac{{c}_{i}}{{c}_{i , S}} = \frac{R}{x} \frac{sinh ( {φ}_{SILP} \frac{x}{R} )}{sinh ( {φ}_{SILP} )} ,$$
where φ_{SILP} is a dimensionless number in respect of the spherical catalyst pellet relating the mass transfer, in the pore, across the phase interface between gas and ionic liquid and in the ionic liquid to the reaction.

2. Process according to claim 1, characterized in that
$${φ}_{SILP} = R \sqrt{\frac{\sqrt{{kD}_{i}^{IL}} a}{{D}_{eff} {H}_{12}}}$$
where
k=the reaction rate constant,
Dᵢ^{IL}=the diffusion coefficient of component i in the ionic liquid,
α=the loading with ionic liquid,
D_{eff}=the effective diffusion coefficient, and
H₁₂=the Henry coefficient.

3. Process according to claim 1, characterized in that
$${φ}_{SILP} = R \sqrt{\frac{2 k {β}_{i} a}{{D}_{eff} {H}_{12} ( k + {β}_{i} a )}}$$
where
k=the reaction rate constant,
α=the loading with ionic liquid,
β=the mass transfer coefficient,
D_{eff}=the effective diffusion coefficient, and
H₁₂=the Henry coefficient.

4. Process according to claim 1, characterized in that
$${φ}_{SILP} = R \sqrt{\frac{k}{{D}_{eff} {H}_{12}}}$$
where
k=the reaction rate constant,
D_{eff}=the effective diffusion coefficient, and
H₁₂=the Henry coefficient.

5. Process according to claim 1, characterized in that the catalyst efficiency of the catalyst pellet is defined by the following relation:
$${η}_{P} = \frac{{r}_{eff}}{r} = \frac{3}{{φ}_{SILP}} ( \frac{1}{tanh ( {φ}_{SILP} )} - \frac{1}{{φ}_{SILP}} ) ,$$
where
ηₚ=the SILP catalyst efficiency,
r=the reaction rate, and
r_{eff}=the effective reaction rate.

6. Process according to claim 1, characterized in that φ_{SILP} is less than 3.

7. SILP catalyst for the hydroformylation of olefins with synthesis gas, wherein the catalyst comprises at least one spherical catalyst pellet of radius R, characterized in that the catalyst complies with the following SILP modulus:
$$\frac{{c}_{i}}{{c}_{i , S}} = \frac{R}{x} \frac{sinh ( {φ}_{SILP} \frac{x}{R} )}{sinh ( {φ}_{SILP} )}$$
where φ_{SILP} is a dimensionless number in respect of the spherical catalyst pellet relating the mass transfer, in the pore, across the phase interface between gas and ionic liquid and in the ionic liquid to the reaction,
where cᵢ is the concentration of a component i and c_{i,S} is the concentration of a component i at the surface of the SILP catalyst pellet, and x is the position.

8. SILP catalyst according to claim 7, characterized in that
$${φ}_{SILP} = R \sqrt{\frac{\sqrt{{kD}_{i}^{IL}} a}{{D}_{eff} {H}_{12}}}$$
where
k=the reaction rate constant,
Dᵢ^{IL}=the diffusion coefficient of component i in the ionic liquid,
α=the loading with ionic liquid,
D_{eff}=the effective diffusion coefficient, and
H₁₂=the Henry coefficient.

9. SILP catalyst according to claim 7, characterized in that
$${φ}_{SILP} = R \sqrt{\frac{2 k {β}_{i} a}{{D}_{eff} {H}_{12} ( k + {β}_{i} a )}}$$
where
k=the reaction rate constant,
α=the loading with ionic liquid,
β=the mass transfer coefficient,
D_{eff}=the effective diffusion coefficient, and
H₁₂=the Henry coefficient.

10. SILP catalyst according to claim 7, characterized in that
$${φ}_{SILP} = R \sqrt{\frac{k}{{D}_{eff} {H}_{12}}}$$
where
k=the reaction rate constant,
D_{eff}=the effective diffusion coefficient, and
H₁₂=the Henry coefficient.

11. SILP catalyst according to claim 7, characterized in that the catalyst efficiency of the catalyst pellet is defined by the following relation:
$${η}_{P} = \frac{{r}_{eff}}{r} = \frac{3}{{φ}_{SILP}} ( \frac{1}{tanh ( {φ}_{SILP} )} - \frac{1}{{φ}_{SILP}} ) ,$$
where
ηₚ=the SILP catalyst efficiency,
r=the reaction rate, and
r_{eff}=the effective reaction rate.

12. SILP catalyst according claim 7, characterized in that φ_{SILP} is less than 3.

13. SILP catalyst according to claim 7, characterized in that the catalyst has at least one ligand having the structure of 2,2′-(3,3′-di-tert-butyl-5,5′-dimethoxybiphenyl-2,2′-diyl)bis(oxy)bis/4,4,5,5-tetraphenyl-1,3,2-dioxaphospholane), also known as benzopinacol.

14. SILP catalyst according to claim 7, characterized in that the catalyst has a transition metal central atom selected from the group comprising rhodium, cobalt, ruthenium, iridium, palladium, platinum and iron.
